(19) United States
(12) Patent Application Publication
(10) Pub. No.: US 2009/0076480 A1
(43) Pub. Date: Mar. 19, 2009

(54) Active Ingredient-Containing Silicone Elastomers

(75) Inventors: Heinz Pudleiner, Krefeld (DE); Ralf Dujardin, Willich (DE); Reinhard Albers, Leverkusen (DE)

Correspondence Address: Baker Donelson Bearman Caldwell & Berkowitz PC;Att: Docketing Sixth Floor, 555 11th Street N.W., Washington, DC, 20004, US

(73) Assignee: Bayer Business Services GmbH, Leverkusen (DE)

(21) Appl. No.: 11/718,587

(22) PCT Filed: Oct. 22, 2005
(86) PCT No.: PCT/EP2005/011365

§ 371 Date: Jan. 25, 2008

(30) Foreign Application Priority Data
Nov. 5, 2004 (DE) 10 2004 054 040.3

(51) Int. Cl. A01N 43/60; A01N 43/42; A61M 25/00; A61L 29/16; A01P 1/00

(52) U.S. Cl. 604/508; 514/311; 514/253.07; 604/544; 604/265

(57) ABSTRACT

The invention relates to compositions comprising silicone elastomers and antimicrobially active substances in homogeneous distribution, a process for their production and their use in medical articles.

The invention relates to compositions comprising silicone elastomers and antimicrobially active substances in homogeneous distribution, to a process for the preparation thereof and to the use thereof in medical articles.

Medical articles made of plastics (e.g. catheters) are currently used in a large number of applications for diagnostic and therapeutic purposes. Central venous catheters are used for example in modern intensive care for invasive monitoring and treatment strategies such as continuous haemofiltration. Urinary catheters are an essential component of modern medical care and are indispensable, for example, in the treatment of impairments of the flow of urine. Although modern medical articles have substantially improved the treatment of intensive-care patients, their application is associated with considerable risks. The frequent use of plastics articles such as, for example, catheters has led to a drastic increase in so-called polymer-associated infections. Catheter-associated infections are in general mainly caused by multiresistant nosocomial pathogens which adhere to the article's plastics surface and then colonize it (Urogenitale Infektionen, Ed. A. Hofstetter, Springer 1999, 241-64).

Catheter-associated infections currently represent an important cause of morbidity and mortality of intensive-care patients. Recent studies demonstrate that 70 to 90% of nosocomially acquired urinary tract infections are associated with an instrumentation (catheterization) of the urinary tract. A single catheterization of the urinary bladder is followed by bacteriuria, for example, in 0.5 to 28% of patients. The incidence of catheter-associated urinary tract infections, moreover, depends on the catheter time and the age, sex and condition (immunocompetence) of the patient (Urogenitale Infektionen, Ed. A. Hofstetter, Springer 1999, 241-64). However, the use of catheters not only involves a higher risk of infection for the patients, but also causes high follow-up therapy costs. Givens and Wenzel were able to show that nosocomial urinary tract infections increase the postoperative inpatient stay by an average of 2.4 days and cause corresponding additional costs (J. Urol. 1980, 124: 646-48). Prevention of catheter-associated infections therefore has the highest priority in modern medicine for both medical and economic reasons.

Catheter-associated infections, possibly developing into sepsis, are, besides traumatic and thromboembolic complications, a serious problem on use of central venous catheters in intensive care.

Numerous studies have revealed that coagulase-negative staphylococci, the transient organism Staphylococcus aureus and various Candida species are the main causes of catheter-associated infections. During application of the catheter, these microorganisms, which are ubiquitously present on the skin, penetrate the physiological barrier of the skin and thus reach the subcutaneous region and eventually the bloodstream. Adhesion of the bacteria to the plastics surface is regarded as an essential step in the pathenogenesis of foreign-body infections. Adhesion of the cutaneous organisms to the polymer surface is followed by the start of metabolically active proliferation of the bacteria with colonization of the polymer. This is associated with production of a biofilm through bacterial excretion of extracellular glycocalix. The biofilm assists adhesion of the pathogens and protects them from attack by certain cells of the immune system. In addition, the film forms a barrier which is impenetrable by many antibiotics. Extensive proliferation of the pathogenic organisms on the polymer surface may finally be followed by septic bacteriaemia. Therapy of such infections requires removal of the infected catheter because chemotherapy with antibiotics would require unphysiologically high doses.

The incidence of bacterially induced infections with central venous catheters averages about 5%. Overall, central venous catheters prove to be responsible for about 90% of all cases of sepsis in intensive care. The use of central venous catheters therefore not only involves a higher risk of infection for the patients, but also causes extremely high follow-up therapy costs (subsequent treatment, extended stays in the clinic).

The problems associated with urinary tract and central venous catheters can be solved only in part by prophylactic measures such as, for example, hygienic measures (handling of the catheters, training of the staff) or routine endoluminal antibiotic administrations.

A rational strategy for preventing polymer-associated infections consists of modifying the polymeric materials used. The aim of this modification must be to inhibit bacterial adhesion and the proliferation of already adherent bacteria, for causal prevention of foreign-body infections in this way. This can be achieved, for example, by incorporating a suitable antimicrobially active substance into the polymer matrix (e.g. antibiotics), provided that the incorporated active ingredient can also diffuse out of the polymer matrix in a controlled manner. An infection-resistant material ought therefore to have the following properties:
1) wide range of effects against the microorganisms relevant for infections associated with the appropriate catheter, especially coagulase-negative staphylococci such as Staphylococcus aureus for central venous catheters and enterococcal, Proteus, Klebsiella, Enterobacter species with urethral catheters
2) sufficient duration of the antimicrobial effect, the requirement being for durations of action of longer than 30 days
3) protection of the internal and external surfaces of the materials
4) polymer modification must not impair either the biocompatibility (thromogenicity, cytotoxicity) or the mechanical properties (tensile strength, modulus, hardness) of the materials

Methods for producing antimicrobially modified polymers for medical applications have already been disclosed.

EP-A 0 696 604 describes aliphatic thermoplastic polyurethane-ureas which are hydrophilic owing to their urea groups but are unable to prevent bacterial adhesion and proliferation on the catheter surface. EP-A 1 067 974, EP-A 0 927 222, EP-A 1 128 724 and EP-A-1 128 723 describe antibacterially effective thermoplastic compounds into which the active ingredients are introduced in sufficiently fine and homogeneous distribution by high viscosity processing techniques. Comparative experiments have shown that the shear forces in the extruder are, however, insufficient to achieve the required distribution of the powdered active ingredients in the silicone solid-phase rubbers employed for producing catheter tubings.

Polymer materials for medical applications which have active ingredient-containing coatings are also mentioned in EP-A 328 421. Descriptions are given of processes for producing the antimicrobially active coatings and methods for application onto the surfaces of medical devices. The coatings consist of a polymer matrix, in particular of polyurethanes, silicones or biodegradable polymers, and of an antimicrobially active substance, preferably of a synergistic combination of a silver salt (silver sulphathiazine) with chlorhexidine or an antibiotic. This publication describes combinations of various polymers, inter alia also silicones, with antibiotics. However, the difficulties of incorporating powdered active ingredients into silicone rubbers are not dealt with. The process according to the invention is not described in this publication.

European Patent EP-A-0 688 564 describes active ingredient-containing silicone elastomers whose delivery rate can be controlled by the density of crosslinking. The special significance of the particle size of active ingredients in silicone elastomers and how this is achieved is not mentioned. In addition, additives which assist the release of active ingredients are described but are deliberately dispensed with in the present invention.

US publication 4 230 686 (Schöpflin et al) describes room temperature-crosslinking (RTV) silicone elastomers which comprise nonionic lipophilic active ingredients. According to this publication (column 5, lines 57 to 59), such silicone elastomers are suitable as active ingredient carriers with slow release only for lipophilic nonionic active ingredients. In addition, column 7, lines 51 to 60, describe the incorporation of the active ingredients as dry powders into the silicone elastomers. The particle size is said in this case to be chosen in such a way that as the solubility of the active ingredient in water increases the size of the incorporated particles (4 to 400 μm) must be larger.

It was an object of the invention to provide novel silicone elastomers which are suitable for producing medical shaped articles for short-term implants, especially catheters, and efficiently prevent for a prolonged period (more than 30 days) surface colonization by microorganisms.

An additional object of the invention was to provide a process making it possible to incorporate active ingredients in fine distribution into silicone elastomers.

It has now surprisingly been found that the silicone elastomers according to the invention which comprise readily water-soluble active ingredients such as, for example, ciprofloxacin hydrochloride with a very small particle size (about 3 μm), and brought about a very good activity against bacterial colonization on catheter surfaces over several weeks.

The present invention accordingly ensures that the active ingredients can be incorporated into the silicone elastomers with particle sizes of from 0.1 to 30 μm, preferably 1 to 20 μm, particularly preferably 2 to 15 μm, particularly preferably 2 to 15 μm.

Silicone elastomers which comprise antimicrobially active substances in homogeneous distribution and which release over a prolonged period (more than 30 days) an antimicrobially active substance on the surface in a concentration which suppresses colonization by organisms have now been found.

The invention thus relates to silicone elastomers and silicone-rubber formulations which comprise an antimicrobially active substance in homogeneous distribution, where the active ingredient is present in particular in the form of a suspension, in an average particle size d₅₀ of from 0.5 to 15 μm, preferably between 1 and 10 μm, and a particle size distribution between 0.1 to 30 μm, preferably 0.5 to 20 μm.

The invention further relates to the use of active ingredient suspensions for incorporating the active ingredient into the silicone-rubber formulation, it being possible in a preferred variant for the suspending medium to be chemically incorporated into the silicone elastomer.

The invention further relates to shaped articles which are produced by crosslinking the silicone-rubber formulations according to the invention at from 150 to 350 C, preferably between 150° C. and 200° C., with retention of the antibacterial activity.

It is known from the literature, e.g. the product brochure “Die platinkatalysierte Additionsvernetzung mit Elastosil R plus” from Wacker, that inter alia amines impair the activity of the platinum catalyst in crosslinking.

It has surprisingly been found that the platinum catalyst retained its activity in the crosslinking of platinum-catalysed silicone-rubber formulations, despite the addition of an active ingredient comprising amine groups. The mechanical properties found for the active ingredient-containing silicone elastomers were the same as for the active ingredient-free comparison specimens.

The invention additionally relates to the use of the active ingredient-containing silicone elastomers for producing medical tubings, urinary bladder catheters (Foley catheters, intermittent catheters, suprapubic and transurethral catheters), haemodialysis catheters, single- and multiple-lumen central venous catheters, peripheral catheters, thermodilution catheters, balloon catheters for percutaneous transluminal coronary angioplasty (PTCA).

The present invention provides active ingredient-containing silicone-rubber formulations which can be crosslinked to give elastomers according to the invention, comprising or consisting of:
A) at least one polysiloxane of the formula (I)

R¹R²₂SiO—(SiR³R⁴O—)ₓSiR¹R²₂ (I)
in which the radicals
R¹ and R² may in each case be identical or different, and are each C₁-C₁₂-alkyl, C₁-C₁₂-fluoroalkyl, and optionally substituted phenyl or naphthyl,
R³ and R⁴ may in each case be identical or different, expressly including each repeating unit, and are each C₁-C₁₂-alkyl, C₁-C₁₂-fluoroalkyl and optionally substituted phenyl or naphthyl, and additionally —OSiR²R³R, in which R symbolizes the continuation of the siloxane chain in analogy to formula (I) in the branching so that the polymer molecule may have branching units of the formula SiO_{4/2} and R³SiO_{3/2},
R¹ and R³ are additionally independently of one another C₁-C₁₂-alkenyl, in which case the polymer comprises from 0.0002 to 3% by weight of vinyl groups, and the molecule has at least two double bonds,
x is an integer from 2 to 15 000 and is varied so that the viscosity of the polymer extends from 0.1 to 1000 Pas at 25° C.,
B) optionally at least one filler having a BET specific surface area of between 50 and 500 m²/g,
C) optionally at least one filler having a BET specific surface area below 50 m²/g,
D) optionally at least one further auxiliary,
E) optionally at least one saturated water repellent from the group consisting of disilazanes, siloxanediols, alkoxysilanes, silylamines, silanols, acetoxysiloxanes, acetoxysilanes, chlorosilanes, chlorosiloxanes and alkoxysiloxanes,
F) optionally at least one unsaturated water repellent from the group consisting of multiply vinyl-substituted methyldisilazanes, and methylsilanols and alkoxysilanes each having unsaturated radicals from the group consisting of alkenyl, alkenylaryl, acryl and methacryl,
G) optionally at least one nonfunctional polysiloxane,
H) optionally at least one inhibitor for the hydrosilylation reaction,
I) at least one polyhydrosiloxane of the formula (II)

R²¹R²²₂SiO—(SiR²³R²⁴O—)ₓSiR²¹R²²₂ (II)
in which the substituents
R²¹ and R²² may in each case be identical or different, and are each C₁-C₁₂-alkyl, C₁-C₁₂-fluoroalkyl, and optionally substituted phenyl or naphthyl,
R²³ in each case expressly including each repeating unit independently of one another is hydrogen, C₁-C₁₂-alkyl, C₁-C₁₂-fluoroalkyl and optionally substituted phenyl or naphthyl, additionally —OSiR²³R²⁴R in which R symbolizes the continuation of the siloxane chain in analogy to formula (II) in the branching so that the polymer molecule may have branching units of the formula SiO_{4/2} and R²³SiO_{3/2}, where R²³ in at least 4 of these silyldioxy units is hydrogen so that a molecule has at least 4 crosslinking sites,
R²⁴ in each case expressly including each repeating unit independently of one another is C₁-C₁₂-alkyl, C₁-C₁₂-fluoroalkyl and optionally substituted phenyl or naphthyl, additionally —OSiR²³R²⁴R in which R symbolizes the continuation of the siloxane chain in analogy to formula (II) in the branching so that the polymer molecule may have branching units of the formula SiO_{4/2} and R²³SiO_{3/2},
x is an integer from 4 to 10 000 and is varied so that the viscosity of the polymer extends from 0.0005 to 0.1 Pas at 25° C.,
J) at least one catalyst comprising an element of the platinum group,
   where a maximum of 3 parts by weight of metal compounds such as oxides and/or carbonates, and further salts and complex compounds, of Fe, Al, Zn, Ti, Zr, Ce or other lanthanoids are present based on 100 parts by weight of component A),
K) at least one active ingredient suspension, where the suspending medium are polysiloxanes of the formula (I) and/or (II) and/or nonfunctional siloxanes G), and comprises at least one active ingredient from the group of
   older quinolones such as, for example, nalixidic acid, pipemidic acid and cinoxacin,
   newer quinolones such as, for example, ciprofloxacin, norfloxacin, ofloxacin, pefloxacin, enoxacin, moxifloxacin, preferably ciprofloxacin, norfloxacin, ofloxacin, particularly preferably ciprofloxacin, their inner salts or hydrochlorides,
   aminoglycosides such as, for example, gentamycin, kanamycin, amikacin, sisomycin, tobramycin, netilmicin, preferably gentamycin and kanamycin, particularly preferably gentamycin, their sulphates or bases,
   polypeptides such as, for example, bacitracin, mupirocin, tyrothricin (combination of gramicidin and tyrocidin),
   lincomycins such as, for example, lincomycin and clindamycin,
   antimycobacterial agents such as, for example, rifampicin
   in an average particle size d₅₀ of from 0.5 to 15 μm, preferably between 1 and 10 μm, and a particle size distribution between 0.1 to 30 μm, preferably 0.5 to 20 μm.

“Expressly including each repeating unit” means that, in a deviation from the exact definition of the corresponding formula, that, for example, in the stated repeating units of the polymers employed according to the invention, of the formula (I), each individual R³ or R⁴ which occurs x times in one molecule can be selected in each case independently from the stated definitions and their preferred ranges, i.e. the radicals occurring in one molecule may be identical or different.

It is possible in principle to use, apart from the silicone-rubber formulations which are described herein as polymer matrix and undergo platinum-catalysed crosslinking at room temperature, also heat-vulcanizable (HV) formulations which are vulcanized at temperatures of about 200° C. with vulcanization catalysts based on benzoyl peroxide or di-p-chlorobenzoyl peroxide and require a thermal aftertreatment. Such silicone elastomers can be produced as described in U.S. Pat. Nos. 2,541,137 or 3,002,951.

The silicone rubbers which undergo platinum-catalysed crosslinking at room temperature are preferred in the present invention because the active ingredients employed might be chemically changed in the case of HV silicone-rubber systems at the required high vulcanization temperature and with use of peroxide catalysts. In addition, the catalyst residues which remain in the elastomer in the case of HV silicone-rubber systems might be responsible for toxic reactions in the body.

In addition, so-called single-component silicone-rubber formulations which are cured at room temperature on exposure to atmospheric humidity without further addition are used. These single-component formulations comprise mainly organopolysiloxanes having two terminal acyloxy, such as, for example, acetoxy, groups which hydrolyse on exposure to atmospheric humidity with formation of trifunctional siloxane units and act in the polymer as crosslinkers with formation of elastomers.

The acetic acid eliminated from usual moisture-curing silicone-rubber formulations as byproduct of the vulcanization at room temperature on exposure to atmospheric humidity may undergo unwanted side reactions with the active ingredient employed.

In a preferred embodiment, the invention therefore relates to crosslinkable active ingredient-containing silicone-rubber formulations in which
the polysiloxane A) is a polysiloxane of the formula (I)

R¹R²₂SiO—(SiR³R⁴O—)SiR¹R²₂ (I)

in which the radicals
R¹ and R² may in each case be identical or different, and are each C₁-C₁₂-alkyl, C₁-C₁₂-fluoroalkyl, and optionally substituted phenyl or naphthyl,
R³ and R⁴ may in each case be identical or different, expressly including each repeating unit, and are each C₁-C₁₂-alkyl, C₁-C₁₂-fluoroalkyl and optionally substituted phenyl or naphthyl,
R¹ and R³ are additionally independently of one another C₁-C₁₂-alkenyl, in which case the polymer comprises from 0.0002 to 3% by weight of vinyl groups, and the molecule has at least two double bonds,
x is an integer from 2 to 15 000 and is varied so that the viscosity of the polymer extends from 0.1 to 1000 Pas at 25° C.,
a filler B) having a BET specific surface area of between 50 and 400 m²/g,
the polyhydrosiloxane I) corresponds to the formula (II)

R²¹R²² ₂SiO—(SiR²³R²⁴O—)SiR²¹R²²₂ (II)

in which the substituents
R²¹ and R²² may in each case be identical or different, and are each C₁-C₁₂-alkyl, C₁-C₁₂-fluoroalkyl, and optionally substituted phenyl or naphthyl,
R²³ in each case expressly including each repeating unit independently of one another is hydrogen, C₁-C₁₂-alkyl, C₁-C₁₂-fluoroalkyl and optionally substituted phenyl or naphthyl, where R²³ is hydrogen in at least 4 of these silyldioxy units so that a molecule has at least 4 crosslinking sites,
R²⁴ in each case expressly including each repeating unit independently of one another is C₁-C₁₂-alkyl, C₁-C₁₂-fluoroalkyl and optionally substituted phenyl or naphthyl,
x is an integer from 4 to 10 000 and is varied so that the viscosity of the polymer extends from 0.0005 to 0.1 Pas at 25° C.,
the catalyst from the platinum group J) is a catalyst which catalyses the hydrosilylation reaction and is selected from metals of the platinum group such as Pt, Rh, Ni, Ru, and compounds of metals of the platinum group, such as salts or complex compounds thereof,
the suspending medium used for the active ingredient suspension K) is at least one polysiloxane of the formula (I) according to A) in which the substituents R¹ to R⁴ are each methyl and vinyl radicals, so that the polymer comprises from 0.0002 to 3% by weight of vinyl groups, and the molecule has at least two double bonds, and x is varied so that the viscosity of the polymer extends from 0.1 to 1000 Pas at 25° C.,
and at least one of the active ingredients from the group of
   older quinolones such as, for example, nalixidic acid, pipemidic acid and cinoxacin,
   newer quinolones such as, for example, ciprofloxacin, norfloxacin, ofloxacin, pefloxacin, enoxacin, moxifloxacin, preferably ciprofloxacin, norfloxacin, ofloxacin, particularly preferably ciprofloxacin, their inner salts or hydrochlorides,
   aminoglycosides such as, for example, gentamycin, kanamycin, amikacin, sisomycin, tobramycin, netilmicin, preferably gentamycin and kanamycin, particularly preferably gentamycin, their sulphates or bases, and
this comprises in each case in an average particle size d₅₀ of from 0.5 to 15 μm, preferably between 1 and 10 μm, and a particle size distribution between 0.1 to 30 μm, preferably 0.5 to 20 μm.

C₁-C₁₂-Alkyl for the purposes of the present invention are expediently aliphatic hydrocarbon radicals having 1 to 12 carbon atoms, which may be straight-chain or branched. Examples which may be listed are methyl, ethyl, propyl, n-butyl, pentyl, hexyl, heptyl, nonyl, decyl, isopropyl, neopentyl, and 1,2,3-trimethylhexyl.

C₁-C₁₂-Fluoroalkyl means for the purposes of the present invention aliphatic hydrocarbon radicals having 1 to 12 carbon atoms, which may be straight-chain or branched and are substituted by at least one fluorine atom.

Examples which may be listed are perfluoroalkyle, 1,1,1-trifluoropropyl, 1,1,1-trifluorobutyl, and trifluoropropyl is preferred.

Substituted phenyl means for the purposes of the present invention phenyl radicals which are unsubstituted or mono- or polysubstituted by F, Cl, CF₃, C₁-C₆-alkyl, C₁-C₆-alkoxy, C₃-C₇-cycloalkyl, C₂-C₆-alkenyl or phenyl; phenyl is preferred.

For the purposes of the present invention, component A) is defined by at least one linear or branched polysiloxane of the general formula (I) indicated hereinbefore.
R¹ and R² may in each case be identical or different, and each is preferably C₁-C₁₂-alkyl, C₁-C₁₂-fluoroalkyl, and phenyl or naphthyl which is optionally mono- or polysubstituted by F, Cl, CF₃, C₁-C₆-alkyl, C₁-C₆-alkoxy, C₃-C₇-cycloalkyl, C₂-C₆-alkenyl or phenyl.
R³ and R⁴ may in each case be identical or different, expressly including each repeating unit, and are each preferably C₁-C₁₂-alkyl, C₁-C₁₂-fluoroalkyl and phenyl or naphthyl which is optionally mono- or polysubstituted by F, Cl, CF₃, C₁-C₆-alkyl, C₁-C₆-alkoxy, C₃-C₇-cycloalkyl, C₂-C₆-alkenyl or phenyl.
R¹ and R³ are preferably in addition independently of one another also C₁-C₁₂-alkenyl, where the polymer comprises from 0.0002 to 3% by weight of vinyl groups, and each molecule has at least two double bonds.
x is preferably an integer from 2 to 15 000 and is varied so that the viscosity of the polymer extends from 0.1 to 1000 Pas at 25° C.
R² to R⁴ are particularly preferably C₁-C₁₂-alkyl.
R¹ is particularly preferably vinyl.
R² to R⁴ are very particularly preferably methyl.

The viscosity of component A) is preferably between 0.1 and 30 000 Pa·s.

For the purposes of the present invention, component B) has the meaning of a filler having a BET specific surface area of between 50 and 500 m²/g. It is expedient for these to be reinforcing fillers. Reinforcing means in this connection that the mechanical strength properties are improved, in particular tear propagation resistance, etc. are improved. The reinforcing fillers are expediently added in a form which positively influences or at least does not impair the electrical properties of the cured mixtures according to the invention. This is achieved for example by addition of precipitated or pyrogenic, preferably pyrogenic, silica having a BET surface area of from 50 to 500 m²/g (the BET surface area is determined by the method of S. Brunauer, P. H. Emmett, E. Teller, J. Am. Soc. 60, 309 (1938)).

The fillers may be hydrophobic or hydrophilic fillers. The fillers B) may be surface-modified, i.e. made water-repellent, e.g. with organosilicon compounds. The modification can take place before or else during the compounding for the silicon-rubber formulation according to the invention.

Components E) and/or F) are preferably used for making water-repellent where appropriate with addition of water. Saturated or unsaturated disilazanes and methylsilanols, which may where appropriate also be produced from the disilazanes, in accordance with the definition of components E) or F) are preferably used for making water-repellent.

Preferred ranges for the BET surface area of the filler B) are from 50 to 400, particularly 150 to 300, m²/g. The amount of component B) is expediently between 0 and 75 parts by weight per 100 parts by weight of component A), preferably 20 to 50 parts by weight.

For the purposes of the present invention, component C) is at least one filler having a BET specific surface area of below 50, preferably below 40, more preferably below 30, m²/g. So-called “non-reinforcing fillers” which do not improve the mechanical properties, in particular the tensile strength, tear propagation resistance, etc., are expedient. Preference is given to diatomaceous earths, finely ground quartz or cristobalite, other amorphous silicas or silicates. The amount of component C) is expediently between 0 and 300 parts by weight per 100 parts by weight of component A), preferably 0 to 50 parts by weight.

For the purposes of the present invention, the term “auxiliary” according to component D) expediently includes pigments, release agents, extrusion aids and hot-air stabilizers, i.e. stabilizers against hot-air aging. The release agents are expediently selected from the group of mold release agents such as, for example, stearyl derivatives or waxes, metal salts of fatty acids. Extrusion agents are, for example, boric acid or PTFE pastes. Hot-air stabilizers are, for example, metal compounds such as oxides and/or carbonates, and further salts and complex compounds, of Fe, Al, Zn, Ti, Zr, Ce or other lanthanoids and antioxidants. The amount of component D) is expediently between 0 and 10 parts by weight per 100 parts by weight of component A), excluding the presence of more than 3 parts by weight, preferably more than 2 parts by weight, of metal compounds, such as oxides and/or carbonates, and further salts and complex compounds, of Fe, Al, Zn, Ti, Zr, Ce or other lanthanoids.

The silicone formulation according to the invention preferably comprises no metal compounds such as oxides and/or carbonates and no further salts and complex compounds of Fe, Al, Zn, Ti, Zr, Ce or other lanthanoids.

For the purposes of the present invention, component E) is at least one saturated water repellent from the group consisting of disilazanes, siloxanediols, alkoxysilanes, silylamines, silanols, acetoxysiloxanes, acetoxysilanes, chlorosilanes, chlorosiloxanes and alkoxysiloxanes. Component E) serves to make the fillers C) preferably B) water-repellent. The making water-repellent can moreover take place separately before the compounding or in situ during the compounding. The amount of component E) is expediently from 0 to 30 parts by weight, preferably 2 to 25, based on 100 parts by weight of B).

For the purposes of the present invention, component F) is at least one unsaturated water repellent from the group consisting of multiply vinyl-substituted methyldisilazanes, and methylsilanols and alkoxysilanes each having unsaturated radicals from the group consisting of alkenyl, alkenylaryl, acryl and methacryl. Component F) likewise serves to make the fillers B) and C) water-repellent. The amount of component F) is expediently from 0 to 2 parts by weight, preferably 0.01 to 1, based on 100 parts by weight of A).

The total amount of components E) and F) is preferably 5-25% by weight based on the total amount of components B) and C), preferably based on B).

For the purposes of the present invention, the term “non-functional polysiloxanes” according to component G) expediently means low molecular weight polysiloxanes which are non-functional in relation to the hydrosilylation reaction, are non-crosslinkable, are preferably trimethylsilyl end-blocked and have dimethyl-, diphenyl or phenylsilyloxy groups with degrees of polymerization of 4-1000, which after crosslinking reliably make the surface of the shaped article hyrophobic, as described for example in EP-A 0 057 098. The amount of component G) is expediently from 0 to 15, preferably 1 to 3, parts by weight based on 100 parts by weight of A).

For the purposes of the present invention, the term “inhibitor for the hydrosilylation reaction” according to component H) includes all inhibitors known in the art for the hydrosilylation reaction with metals of the Pt group, such as, for example, maleic acid and its derivatives, amines, azoles, alkylisocyanurates, phosphines, phosphites and acetylenically unsaturated alcohols in which the OH group is bonded to the carbon atom adjacent to the C—C triple bond, as are described in detail for example in U.S. Pat. No. 3,445,420. Component G) is preferably 2-methyl-3-butyn-2-ol or 1-ethynylcyclohexanol or (±)-3-phenyl-1-butyn-3-ol. Component H) is preferably used in a proportionate amount of from 0 to 1 parts by weight based on 100 parts by weight of the total of A) to I). Component H) is preferably present in a proportionate amount of from 0.0001% to 2% by weight, particularly preferably 0.01% by weight to 2% by weight and very particularly preferably 0.05% by weight to 0.5% by weight, in each case based on the total weight of the mixture.

For the purposes of the present invention, component I) is defined by at least one polyhydrosiloxane which has at least two hydrogen atoms directly linked to different silicon atoms, according to general formula (II) indicated hereinbefore. The following definitions apply to the radicals therein:
R²¹ and R²² may in each case be identical or different, and are preferably each C₁-C₁₂-alkyl, C₁-C₁₂-fluoroalkyl, and optionally substituted phenyl or naphthyl.
R²³ is preferably in each case expressly including each repeating unit independently of one another hydrogen, C₁-C₁₂-alkyl, C₁-C₁₂-fluoroalkyl and optionally substituted phenyl or naphthyl, where R²³ is hydrogen in at least 4 of these silyldioxy units so that a molecule has at least 4 crosslinking sites.
R²⁴ is in each case expressly including each repeating unit independently of one another C₁-C₁₂-alkyl, C₁-C₁₂-fluoroalkyl and optionally substituted phenyl or naphthyl.
x is preferably an integer from 4 to 10 000 and is varied so that the viscosity of the polymer extends from 0.0005 to 0.1 Pas at 25° C.

The molar proportion of hydrogen atoms directly linked to a silicon atom in component I) is preferably between 0.01 and 10 mmol/g, particularly preferably between 0.5 and 9 mmol/g and very particularly preferably between 1 and 7, mmol/g.

The amount of component I) is preferably from 0.2 to 30, particularly preferably 0.2 to 20, parts by weight based on 100 parts by weight of component A).

Component J) is a catalyst at least comprising one element of the platinum group. Component J) is preferably a catalyst which catalyses the hydrosilylation reaction and is selected from metals of the platinum group such as Pt, Rh, Ni, Ru and compounds of metals of the platinum group, such as salts or complex compounds thereof. It is further preferred for component J) to be a catalyst comprising an element from the platinum group selected from platinum and platinum compounds, which may optionally be adsorbed on a support, and other compounds of elements of the platinum group. Platinum and platinum compounds are most preferred. Thus, Pt salts, Pt complex compounds with nitrogen, phosphorus compounds and/or alkene compounds or Pt metals on supports are preferably employed. All Pt(0) and Pt(II) compounds are preferred, and Pt-olefin complexes and Pt-vinylsiloxane complexes are particularly preferred. Pt-Vinylsiloxane complexes, Pt-vinyldi- and tetrasiloxane complexes, which preferably have at least 2 or 4 olefinically unsaturated double bonds, are particularly preferred (see, for example, U.S. Pat. No. 3,715,334). The term siloxane includes in this connection polysiloxanes or else polyvinylsiloxanes.

It is additionally possible for component J) also to be a product of the reaction of reactive platinum compounds with the inhibitors H).

The amount of component J) in the formulation according to the invention is preferably from 10 to 100 ppm, particularly preferably 15 to 80 ppm and very particularly preferably 20 to 50 ppm, based on the total amount of components A) to I) and calculated on the basis of the metal of the platinum group in component J). The silicone-rubber formulations preferably comprises 20-100 ppm Pt, based on the amount of components A) to J), in the form of Pt salts, Pt complex compounds with nitrogen compounds, phosphorus compounds and/or alkene compounds or Pt metal on supports.

The active ingredient suspension K) consists on the one hand preferably of polysiloxanes of formula (I) indicated hereinbefore as suspending agents. The definitions of the radicals therein are as follows
R¹ to R⁴ are independently of one another particularly preferably each methyl and vinyl, where the polymer comprises from 0.0002 to 3% by weight of vinyl groups, and each molecule has at least two double bonds.
x is particularly preferably varied so that the viscosity of the polymer extends from 0.1 to 1000 Pas at 25° C.

The active ingredient suspension K) comprises on the other hand preferably active ingredients from the group of
older quinolones such as, for example, nalixidic acid, pipemidic acid and cinoxacin,
newer quinolones such as, for example, ciprofloxacin, norfloxacin, ofloxacin, pefloxacin, enoxacin, moxifloxacin, preferably ciprofloxacin, norfloxacin, ofloxacin, particularly preferably ciprofloxacin, their inner salts or hydrochlorides,
aminoglycosides such as, for example, gentamycin, kanamycin, amikacin, sisomycin, tobramycin, netilmicin, preferably gentamycin and kanamycin, particularly preferably gentamycin, their sulphates or bases,

dispersed in an average particle size d₅₀ of from 0.5 to 15 μm, preferably between 1 and 10 μm, and a particle size distribution between 0.1 to 30 μm, preferably 0.5 to 20 μm.

The powdered active ingredients are usually supplied in micronized form. In order to incorporate them into the silicone rubbers, they are previously suspended in a suitable medium. Care must be taken in this connection that the medium has good solubility in the silicone elastomer. Suitable for this purpose in one embodiment of the invention are commercially available silicone oils (R′ and R″ equal to alkyl), vinyl-terminated polydimethylsiloxanes (R′ equal vinyl; R″ equal methyl) or polyhydrosiloxanes (R′ equal H; R″ equal methyl), which have viscosities of from 100 to 1 000 000 mPas, preferably from 100 to 500 000 mPas at 25° C. The suitability is decided by whether the active ingredient/medium mixture can be sufficiently finely homogenized in a bead mill.

In a preferred variant, the suspending medium used is at least one vinyl group-terminated silicone polymer which is chemically incorporated into the silicone elastomer in the subsequent crosslinking reaction. It is thereby no longer possible for the suspending medium to be leached out into the surrounding body tissue or a body fluid on use of the silicone elastomer. For example, vinyl group-terminated silicone polymers are available as polymer VS 200 (η(25° C.)=200 mPas; vinyl group content 0.25 mmol/g), polymer VS 1000 (η(25° C.)=1000 mPas; vinyl group content 0.11 mmol/g), or polymer VS 165 000 (η(25° C.)=165 000 mPas; vinyl group content 0.015 mmol/g), from Hanse-Chemie. Comparable products are available from other suppliers such as Dow Corning (Syl-Off® 7673: η(25° C.)=425 mPas) or Wacker Silicones (Dehesive® 920; η(25° C.)=500 mPas or Dehesive® 924; η(25° C.)=200 mPas).

Suitable antimicrobially active substances are in principle all active ingredients which have a wide range of effects against the pathogenic microorganisms involved in polymer-associated infections. In the case of central venous catheters these are in particular substances which are effective against coagulase-negative staphylococci, Staphylococcus aureus and Candida species. The antimicrobially active substances may according to the invention also be used as active ingredient combinations in the shaped articles as long as their effects are not mutually antagonistic. In the case of urine catheters, the pathogenic microorganisms involved in polymer-associated infections are in particular enterococcal, Proteus, Klebsiella, Enterobacter species.

The active ingredient used must have an adequate (chemical) stability in the silicone-rubber matrix. In addition, the microbiological activity of the active ingredient must not be impaired in the polymeric matrix and under the processing conditions for incorporation and subsequent thermal crosslinking, and the active ingredient must therefore be stable at the temperatures of from 150 to 350° C., preferably between 150° C. to 200° C., which are necessary for the thermal crosslinking of the silicone rubber.

In addition, the active ingredient must not reduce the activity of the platinum catalyst used for the crosslinking reaction of room temperature-crosslinking 2K silicone rubbers. Inadequately crosslinked silicone elastomers may still comprise monomers which are then responsible for cytotoxic reactions of the material. Accordingly, the incorporation of the pharmaceutically active substance must not impair either the biocompatibility of the polymer surface or other desirable polymer-specific properties of the silicone elastomer (elasticity, tensile strength etc.).

Examples of suitable antibiotically active substances are
older quinolones such as, for example, nalixidic acid, pipemidic acid and cinoxacin,
newer quinolones such as, for example, ciprofloxacin, norfloxacin, ofloxacin, pefloxacin, enoxacin, moxifloxacin, preferably ciprofloxacin, norfloxacin, ofloxacin, particularly preferably ciprofloxacin, their inner salts or hydrochlorides,
aminoglycosides such as, for example, gentamycin, kanamycin, amikacin, sisomycin, tobramycin, netilmicin, preferably gentamycin and kanamycin, particularly preferably gentamycin, their sulphates or bases,
macrolides such as, for example, erythromycin, clarithromycin and azithromycin,
polypeptides such as, for example, bacitracin, mupirocin, tyrothricin (combination of gramicidin and tyrocidin),
lincomycins such as, for example, lincomycin and clindamycin,
antimycobacterial agents such as, for example, rifampicin or
fusidic acid

The antimicrobially active substance may also be an antiseptic or a disinfectant as long as the substance used has sufficient activity against the infection-causing species.

Preference is given to newer quinolones such as, for example, ciprofloxacin, norfloxacin, ofloxacin, perfloxacin, enoxacin, moxifloxacin, particularly preferably ciprofloxacin, norfloxacin, ofloxacin, their inner salts or hydrochlorides, and mixtures thereof.

It is additionally possible to employ substances (prodrugs) which release an antimicrobially active substance after the influence of microbial activity.

The active ingredients are preferably incorporated into the silicone formulations according to the invention in a concentration appropriate for their antimicrobial activity. The active ingredients are used in a concentration range of from 0.01 to 10.0% by weight, preferably 0.05 to 5% by weight, particularly preferably 0.1 to 5% by weight, in the silicone elastomers.

Embodiments which are preferred, particularly preferred or very particularly preferred are those which make use of the parameters, compounds, definitions and explanations which are specified as preferred, particularly preferred or very particularly preferred.

However, the general definitions, parameters, compounds and explanations mentioned in the description, or definitions, parameters, compounds and explanations mentioned in preferred ranges, may also be combined with one another, that is to say between the respective ranges and preferred ranges, as desired.

Polymeric additives such as polyvinylpyrrolidone or polyethylene glycol can in principle be admixed to the silicone rubbers up to a concentration of 5% by weight. In a preferred embodiment, such additives influencing the release to the surface are dispensed with.

The suspension K) is produced by using conventional dissolvers which are employed as bead mill. Active ingredient, suspending medium and beads are put into the temperature-controlled vessel. In addition to the total volume, ⅓ glass beads are also added. Instead of glass beads, it is also possible to use other grinding beads, e.g. made of zirconium oxide.

The concentration of the active ingredient in the suspension K) is from 10 to 40% by weight, preferably 15 to 35% by weight. The material for grinding can be heated to up to 100° C. in order to adjust the viscosity suitable for the grinding. However, the lowest possible temperature is always to be preferred in order to carry out the processing of the active ingredient under conditions which are as mild as possible.

The suspensions K) are incorporated into the silicone-rubber matrix, for example, on a roll mixer. Their viscosity must not be too low for this purpose because they flow away too easily. The risk associated with pastes which are too viscous is that they cannot be incorporated homogeneously into the silicone rubber.

The suspensions K) according to the invention therefore ought to have viscosities of from 10 000 mPas to 2 000 000 mPas at room temperature. Those preferably suitable for use for the process according to the invention have viscosities at 25° C. of from 20 000 to 1 000 000 mPas, particularly preferably from 50 000 to 500 000 mPas.

The active ingredients in the suspension K) according to the invention usually have an average particle size d₅₀ of from 0.5 to 15 μm, preferably between 1 and 10 μm, and a particle size distribution between 0.1 to 30 μm, preferably 0.5 to 20 μm.

In addition, the suspensions K) produced in this viscosity range remain stable for several weeks and do not sediment. It is possible to dispense with additional dispersion aids.

Components A)+F)+K) and I) ought preferably to be present in the active ingredient-containing silicone-rubber mixtures according to the invention in the ratio of amounts such that the molar ratio of hydrogen directly linked to a silicon atom (SiH) in component I) to unsaturated radicals in components A), F) and K) is between 0.1 and 20, preferably between 0.8 and 10 and very particularly preferably between 1 and 5.

The active ingredient-containing silicone-rubber formulations according to the invention consist of components A) to K), with components B) to H) being optionally present. The silicone-rubber formulation according to the invention preferably comprises component G) in addition to the necessary components A), I), J) and K).

In the rubber formulations according to the invention it is possible for ingredients A), polysiloxanes of the formula (I), and I), polyhydrosiloxanes of the formula (II), to be present completely or partly in component K), the active ingredient suspension, as suspending medium. Also included here according to the invention are formulations without separate further components A) and/or I).

The invention further relates to a process for producing the silicone-rubber formulations according to the invention, which is characterized in that initially components A) to J) are combined and mixed, and K) is then added and incorporated.

The active ingredient suspension K) is added to the silicone-rubber compositions on a roll mill, in a kneader or on an extruder. In a preferred embodiment, in the case of 2-component systems the two components are premixed and then the active ingredient suspension is added.

The silicone-rubber formulations according to the invention are preferably produced by adding the water repellents E) and F) which are optionally used, and optionally water, to component A), and incorporating component D) (filler) at temperatures of from 20 to 160° C. under a nitrogen atmosphere, and thus making the filler D) water-repellent by reaction with components E) and F). Subsequently, excess reaction products E) and F), and volatile reaction products therefrom (such as silanols, alcohols and water) are removed (preferably by heating at 150 to 170° C., where appropriate in vacuo). In the case of a 2-component formulation, either component H) and I) or alternatively J) is metered into the resulting, preferably cooled mixture. If components C), D) and G) are required, they are metered after removal of the volatile components E) and F). In the case of the single-component formulation, H), I) and J) are metered in, the inhibitor H) being metered in first.

Conventional mixers are used, such as, for example, internal mixers, screw mixers, kneaders, preferably kneaders.

The crosslinkable silicone-rubber compositions according to the invention may moreover be 1-, 2- or else multicomponent systems. Multicomponent systems are for example those which comprise H), I) and J) separately.

The following examples serve to illustrate the invention without having a limiting effect.

### Raw Materials:

### Silicone Solid Rubbers

A 50:50 A/B 2K platinum-catalysed solid silicone-rubber system 3097/PA from Degania was used for the experiments.

A component: vinyl group-terminated polydimethylsiloxane; comprises ingredients A), B) and J).

B component: polyhydrosiloxane; comprises components B), G) and I).

The ratios of the amounts of ingredients A), B), G), I) and J) are adjusted in the A/B components so that the silicone elastomer has a Shore A hardness of 65.

### Liquid Rubbers

Silopren H60: vinyl group-terminated polydimethylsiloxane from GE Bayer Silicones; viscosity (at 25° C.)=60 000 mPas; vinyl content: 0.20 mmol/g.

Silopren H6: vinyl group-terminated polydimethylsiloxane from GE Bayer Silicones; viscosity (at 25° C.)=6000 mPas; vinyl content: 0.22 mmol/g.

Crosslinker 930: polyhydrosiloxane from GE Bayer Silicones; SiH content=9.3 mmol/g, viscosity (at 25° C.)=35 mPas.

### Suspending Media

Polymer VS 200: vinyl group-terminated polydimethylsiloxane from Hanse-Chemie; viscosity (at 25° C.)=200 mPas; vinyl content: 0.25 mmol/g

Polymer VS 1000: vinyl group-terminated polydimethylsiloxane from Hanse-Chemie; viscosity (at 25° C.)=1000 mPas; vinyl content: 0.11 mmol/g

Baysilon M 100: nonfunctional, non-crosslinkable trimethylsilyl end-blocked polysiloxane

### Active Ingredients

Ciprofloxacin hydrochloride: Bayer HealthCare AG, Wuppertal, white powder with an average particle diameter of d₅₀=48 μm

Ciprofloxacin: Bayer HealthCare AG, Wuppertal, white powder with an average particle diameter of d₅₀=14 μm

### EXAMPLES 1-3

### Production of the Active Ingredient Suspension in Polymer VS 1000

A Dispermat F 105 dissolver from VMA Getzmann was used to produce the suspension. A plastic disc was used as grinding tool. The temperature of the temperature-controlled vessel was controlled using a thermostat from Julabo HC.

45 g of vinyl-terminated silicone polymer VS 1000, 15 g of ciprofloxacin hydrochloride, ciprofloxacin or mixtures thereof (see table) and 20 ml of glass beads with a diameter of 3 mm are weighed into a 250 ml temperature-controlled vessel. The temperature of the vessel is controlled at 25° C., and the dissolver is started up. The material to be ground is mixed at 10 000/min for 30 minutes. The glass beads are then removed. A white creamy paste is obtained. The average particle diameter was determined.

| | | | Average particle |
|---|---|---|---|
| | Active ingredients | Concentration | size d₅₀ |
| Example 1 | Hydrochloride:betaine | 25% by weight | 3.5 μm |
| | ratio ⅓ to ⅔ | | |
| Example 2 | Hydrochloride:betaine | 25% by weight | 3.2 μm |
| | ratio ½ to ½ | | |
| Example 3 | Hydrochloride:betaine | 25% by weight | 4.3 μm |
| | ratio ⅔ to ⅓ | | |

Explanation: Hydrochloride: ciprofloxacin hydrochloride; betaine: ciprofloxacin

### EXAMPLES 4-6

### Production of the Active Ingredient Suspension in Polymer VS 200

A Dispermat F 105 dissolver from VMA Getzmann was used to produce the suspension. A plastic disc was used as grinding tool. The temperature of the temperature-controlled vessel was controlled using a thermostat from Julabo HC.

45 g of vinyl-terminated silicone polymer VS 200, 15 g of ciprofloxacin hydrochloride, ciprofloxacin or mixtures thereof (see table) and 20 ml of glass beads with a diameter of 3 mm are weighed into a 250 ml temperature-controlled vessel. The temperature of the vessel is controlled at 25° C., and the dissolver is started up. The material to be ground is mixed at 10 000/min for 30 minutes. The glass beads are then removed. A white creamy paste is obtained. The average particle diameter was determined.

| | | | Average particle |
|---|---|---|---|
| | | Concentration | size d₅₀ |
| Example 4 | Hydrochloride:betaine | 25% by weight | 5.5 μm |
| | ratio ⅓ to ⅔ | | |
| Example 5 | Hydrochloride:betaine | 25% by weight | 5.4 μm |
| | ratio ½ to ½ | | |
| Example 6 | Hydrochloride:betaine | 25% by weight | 6.4 μm |
| | ratio ⅔ to ⅓ | | |

Explanation: Hydrochloride: ciprofloxacin hydrochloride; betaine: ciprofloxacin

### EXAMPLES 7-9

### Production of the Active Ingredient Suspension in Baysilon M 100

A Dispermat F 105 dissolver from VMA Getzmann was used to produce the suspension. A plastic disc was used as grinding tool. The temperature of the temperature-controlled vessel was controlled using a thermostat from Julabo HC.

45 g of Baysilon M 100 silicone oil, 15 g of ciprofloxacin hydrochloride, ciprofloxacin or mixtures thereof (see table) and 20 ml of glass beads with a diameter of 3 mm are weighed into a 250 ml temperature-controlled vessel. The temperature of the vessel is controlled at 25° C., and the dissolver is started up. The material to be ground is mixed at 10 000/min for 30 minutes. The glass beads are then removed. A white creamy paste is obtained. The average particle diameter was determined.

| | | | Average |
|---|---|---|---|
| | | Concentration | particle size d₅₀ |
| Example 7 | Hydrochloride:betaine ratio | 12.5% by | 3.8 μm |
| | ⅓ to ⅔ | weight | |
| Example 8 | Hydrochloride:betaine ratio | 12.5% by | 4.5 μm |
| | ½ to ½ | weight | |
| Example 9 | Hydrochloride:betaine ratio | 12.5% by | 4.3 μm |
| | ⅔ to ⅓ | weight | |

Explanation: Hydrochloride: ciprofloxacin hydrochloride; betaine: ciprofloxacin

### EXAMPLES 10

### Production of the Active Ingredient Suspension in Silopren H 60 (Without Beads)

A Dispermat F 105 dissolver from VMA Getzmann was used to produce the suspension. A dissolver disc was used as grinding tool. The temperature of the temperature-controlled vessel was controlled using a thermostat from Julabo HC.

45 g of Silopren H 60 silicone oil, 15 g of ciprofloxacin hydrochloride are weighed into a 250 ml temperature-controlled vessel. The temperature of the vessel is controlled at 25° C., and the dissolver is started up. The material to be ground is mixed at 10 000/min for 30 minutes. An inhomogeneous liquid containing coarse white particles is obtained. The particle size determination was not carried out.

### Production of the Active Ingredient-Containing Crosslinkable Silicone-Rubber Mixtures

### EXAMPLES 11-25 (ACCORDING TO THE INVENTION)

### 26-28 (Comparative Examples; not According to the Invention)

Equal parts (see table) of each of the A and B solid silicone-rubber components were mixed together at room temperature with cooling in a roll mixer from Vogt (2 rolls; roll diameter 80 mm, roll width 280 mm; operating width 200 mm). The front rotating roll was operated at 16.5 min⁻¹, and the rear roll at 20 min⁻¹. The active ingredients were subsequently incorporated by adding the active ingredient suspension, indicated in the table, from Example 1 to 10 in the roll gap, and continuing the mixing until the suspension was homogeneously incorporated.

| | | | |
|---|---|---|---|
| Amount of | Amount of B | Amount of | Active ingredient |
| A component in g | component in g | suspension | concentration |

Suspension based on polymer VS 1000

| | | | | |
|---|---|---|---|---|
| Example 11 | 48 | 48 | 4 g of suspension | 1% by weight |
| | | | from Example 1 | |
| Example 12 | 46 | 46 | 8 g of suspension | 2% by weight |
| | | | from Example 1 | |
| Example 13 | 48 | 48 | 4 g of suspension | 1% by weight |
| | | | from Example 2 | |
| Example 14 | 46 | 46 | 8 g of suspension | 2% by weight |
| | | | from Example 2 | |
| Example 15 | 48 | 48 | 4 g of suspension | 1% by weight |
| | | | from Example 3 | |
| Example 16 | 46 | 46 | 8 g of suspension | 2% by weight |
| | | | from Example 3 | |

Suspension based on polymer VS 200

| | | | | |
|---|---|---|---|---|
| Example 17 | 48 | 48 | 4 g of suspension | 1% by weight |
| | | | from Example 4 | |
| Example 18 | 46 | 46 | 8 g of suspension | 2% by weight |
| | | | from Example 4 | |
| Example 19 | 48 | 48 | 4 g of suspension | 1% by weight |
| | | | from Example 5 | |
| Example 20 | 46 | 46 | 8 g of suspension | 2% by weight |
| | | | from Example 5 | |
| Example 21 | 48 | 48 | 4 g of suspension | 1% by weight |
| | | | from Example 6 | |
| Example 22 | 46 | 46 | 8 g of suspension | 2% by weight |
| | | | from Example 6 | |

Suspension based on Baysilon M 100

| | | | | |
|---|---|---|---|---|
| Example 23 | 46 | 46 | 8 g of | 1% by weight |
| | | | suspension from | |
| | | | Example 7 | |
| Example 24 | 46 | 46 | 8 g of | 1% by weight |
| | | | suspension from | |
| | | | Example 8 | |
| Example 25 | 46 | 46 | 8 g of | 1% by weight |
| | | | suspension from | |
| | | | Example 9 | |

Comparative examples, not according to the invention

| | | | | |
|---|---|---|---|---|
| Example 26 | 46 | 46 | 8 g of polymer | 0 |
| | | | VS 1000 | |
| Example 27 | 46 | 46 | 8 g of polymer | 0 |
| | | | VS 200 | |
| Example 28 | 46 | 46 | 8 g of Baysilon | 0 |
| | | | M 100 | |

### EXAMPLE 29 (NOT ACCORDING TO THE INVENTION)

49.5 parts of each of the A and B solid silicone-rubber components were mixed together at room temperature with cooling in a roll mixer from Vogt (2 rolls; roll diameter 80 mm, roll width 280 mm; operating width 200 mm). The front rotating roll was operated at 16.5 min⁻¹, and the rear roll at 20 min⁻¹. The active ingredients were incorporated by subsequently adding 1 g of ciprofloxacin hydrochloride as powder in the roll gap, and continuing the mixing until the suspension was homogeneously incorporated.

The mixing process was terminated after 20 minutes: a sheet permeated by coarse white particles was obtained. The active ingredient had been insufficiently dispersed. No further investigations were carried out on the sample.

### Production of the Crosslinked Active Ingredient-Containing Elastomers

After the silicone-rubber mixtures had been mixed on the roll mixer, sheets about 3 mm thick were detached and laid on a Teflon film. The sheets were then vulcanized in a circulating air oven with a supply of fresh air at 180° C. for 2 h.

All the silicone-rubber mixtures resulted in solid, elastic boards which were capable of further processing.

Round discs with a diameter of about 5 mm were cut out of the elastomer boards for the microbiological investigations, and S2 tensile bars complying with DIN ISO 527 were cut out for the mechanical investigations.

### Results of the Mechanical Tests

| | | | |
|---|---|---|---|
| Tensile | Elongation | Stress at 100% | Stress at 300% |
| strength | at break | elongation | elongation |
| [N/mm²] | [%] | [N/mm²] | [N/mm²] |

Suspension based on polymer VS 1000

| | | | | |
|---|---|---|---|---|
| Example 11 | 8.01 | 614.70 | 1.71 | 3.75 |
| Example 12 | 8.72 | 521.08 | 1.88 | 4.53 |
| Example 13 | 9.62 | 607.50 | 1.77 | 4.15 |
| Example 14 | 9.06 | 552.31 | 1.78 | 4.37 |
| Example 15 | 7.57 | 601.33 | 1.63 | 3.65 |
| Example 16 | 8.03 | 628.32 | 1.65 | 3.77 |

Suspension based on polymer VS 200

| | | | | |
|---|---|---|---|---|
| Example 17 | 6.88 | 576.82 | 1.72 | 3.62 |
| Example 18 | 5.71 | 484.63 | 1.89 | 3.87 |
| Example 19 | 7.24 | 616.62 | 1.72 | 3.59 |
| Example 20 | 7.97 | 639.84 | 2.01 | 4.10 |
| Example 21 | 7.88 | 612.75 | 1.89 | 3.92 |
| Example 22 | 7.58 | 614.68 | 1.94 | 3.99 |

Suspension based on Baysilon M 100

| | | | | |
|---|---|---|---|---|
| Example 23 | 8.60 | 668.57 | 1.43 | 3.24 |
| Example 24 | 8.00 | 679.42 | 1.29 | 2.92 |
| Example 25 | 8.34 | 683.20 | 1.33 | 3.04 |

Comparative examples, not according to the invention

| | | | | |
|---|---|---|---|---|
| Example 26 | 7.27 | 504.96 | 1.83 | 4.47 |
| Example 27 | 7.35 | 538.72 | 2.15 | 4.64 |
| Example 28 | 8.36 | 669.10 | 1.36 | 3.23 |

It was surprisingly found that the mechanical properties of the active ingredient-containing silicone elastomers according to the invention do not differ significantly from those of the active ingredient-free comparative samples not according to the invention. It is thus not possible to find an adverse effect of the active ingredients on the activity of the platinum catalyst and thus on the crosslinking reaction.

### Microbiological Tests

The antimicrobial activity of the modified silicone elastomers was tested on the Gram-negative bacterial strains Citrobacter freundii, Enterobacter aerogenes, Enterobacter cloacae, Escherichia coli, Klebsiella oxytoca, Klebsiella pneumoniae, Morganella morganii, Proteus mirabilis, Proteus vulgaris, Pseudomonas aeruginosa and the Gram-positive bacterial strains (29212) Enterococcus faecalis, (29213) Staphylococcus aureus, (25923) Staphylococcus aureus, (1150-93) and (9809) Streptococcus bovis.

TABLE 1
Test of the antimicrobial activity of active ingredient-free and active
ingredient-containing silicone elastomers on various test strains in the agar diffusion
test. The antimicrobial effect is indicated by the formation of a zone of inhibition.
The diameters of the zones of inhibition are reported in mm.

| | | | Hydrochloride | | | | | |
|---|---|---|---|---|---|---|---|---|
| | | | to betaine ratio | | | | | |
| | | | ⅓ to ⅔ | | ½ to ½ | | ⅔ to ⅓ | |
| | | | Concentration in | | | | | |
| | | | % by weight | | | | | |
| | | | 1 | 2 | 1 | 2 | 1 | 2 |
| | | | Example | | | | | |
| | | | 11 | 12 | 13 | 14 | 15 | 16 |
| Gram-neg. | 43864 | Citrobacter freundii | 23 | 28 | 24 | 27 | 25 | 28 |
| | 1304 | Enterobacter aerogenes | 23 | 25 | 24 | 26 | 24 | 26 |
| | 700323 | Enterobacter cloacae | 25 | 28 | 27 | 30 | 27 | 29 |
| | 35218 | Escherichia coli | 23 | 26 | 23 | 28 | 26 | 30 |
| | 25922 | Escherichia coli | 28 | 32 | 28 | 32 | 28 | 32 |
| | 700324 | Klebsiella oxytoca | 28 | 30 | 29 | 30 | 30 | 32 |
| | 13883 | Klebsiella pneumoniae | 26 | 28 | 27 | 29 | 26 | 30 |
| | 25829 | Morganella morganii | 27 | 28 | 27 | 29 | 26 | 28 |
| | 35659 | Proteus mirabilis | 31 | 32 | 30 | 33 | 32 | 34 |
| | 6380 | Proteus vulgaris | 27 | 30 | 29 | 30 | 28 | 30 |
| | 27853 | Pseudomonas aeruginosa | 17 | 19 | 15 | 23 | 19 | 23 |
| Gram.-pos. | 29212 | Enterococcus faecalis | 8 | 13 | 11 | 13 | 10 | 13 |
| | 29213 | Staphylococcus aureus | 18 | 22 | 19 | 21 | 22 | 22 |
| | 25923 | Staphylococcus aureus | 17 | 18 | 16 | 19 | | |
| | 9809 | Streptococcus bovis | 0 | 11 | 8 | 13 | 9 | 13 |

| | | Hydrochloride | | | | | | | | |
|---|---|---|---|---|---|---|---|---|---|---|
| | | to betaine ratio | | | | | | | | |
| | | ⅓ to ⅔ | | ½ to ½ | | ⅔ to ⅓ | | ⅓ to ⅔ | ½ to ½ | ⅔ to ⅓ |
| | | Concentration | | | | | | | | |
| | | 1 | 2 | 1 | 2 | 1 | 2 | 1 | 1 | 1 |
| | | Example | | | | | | | | |
| | | 17 | 18 | 19 | 20 | 21 | 22 | 23 | 24 | 25 |
| Gram-neg. | | | | | | | | | | |
| 43864 | Citrobacter freundii | 25 | 29 | 27 | 29 | 26 | 28 | 22 | 22 | 23 |
| 1304 | Enterobacter | 24 | 27 | 24 | 26 | 24 | 25 | 30 | 30 | 31 |
| | aerogenes | | | | | | | | | |
| 700323 | Enterobacter | 27 | 29 | 28 | 32 | 28 | 30 | 21 | 21 | 21 |
| | cloacae | | | | | | | | | |
| 35218 | Escherichia coli | 24 | 28 | 24 | 27 | 24 | 27 | 25 | 25 | 25 |
| 25922 | Escherichia coli | 28 | 30 | 28 | 32 | 28 | 32 | 24 | 25 | 25 |
| 700324 | Klebsiella oxytoca | 28 | 31 | 29 | 32 | 29 | 32 | 25 | 25 | 25 |
| 13883 | Klbsiella | 27 | 30 | 27 | 29 | 28 | 29 | 19 | 20 | 20 |
| | pneumoniae | | | | | | | | | |
| 25829 | Morganella | 28 | 30 | 28 | 29 | 27 | 29 | 35 | 35 | 35 |
| | morganii | | | | | | | | | |
| 35659 | Proteus mirabilis | 30 | 32 | 29 | 34 | 30 | 35 | 23 | 23 | 23 |
| 6380 | Proteus vulgaris | 29 | 30 | 28 | 32 | 28 | 30 | 22 | 22 | 25 |
| 27583 | Pseudomonas | 18 | 21 | 18 | 26 | 18 | 22 | 17 | 19 | 19 |
| | aeruginosa | | | | | | | | | |
| Gram.-pos | | | | | | | | | | |
| 29212 | Enterococcus | 11 | 13 | 9 | 14 | 9 | 14 | 9 | 12 | 10 |
| | faecalis | | | | | | | | | |
| 29213 | Staphylococcus | 19 | 20 | 17 | 22 | 19 | 24 | 17 | 18 | 19 |
| | aureus | | | | | | | | | |
| 25923 | Staphylococcus | 21 | 25 | 18 | 22 | 18 | 21 | 16 | 18 | 17 |
| | aureus | | | | | | | | | |
| 9809 | Streptococcus bovis | 9 | 13 | 9 | 11 | 8 | 13 | 8 | 8 | 9 |

The bacterial strains were each cultivated in an overnight culture on standard II nutrient agar (from Merck KGaA, D-64293, Darmstadt) and suspended in NaCl solution (0.85%). The resulting suspension of bacteria with a density of 0.5 MacFarland was diluted 1:100 in NaCl solution (0.85%) and applied to agar plates (Mueller-Hinton agar, from Merck KGaA, D-64293 Darmstadt). The polymer samples about 0.2 cm² in size (discs with a diameter of about 5 mm) were gamma-sterilized, placed under slight pressure on the agar plates and incubated at 37° C. for 20 hours. After the incubation, the agar plates were checked for zones of inhibition, and the zones of inhibition were measured.

The results of the agar diffusion test are summarized in table 1. They show that a zone of inhibition in which no bacterial growth takes place was formed around the active ingredient-containing polymer samples compared with the active ingredient-free sample, i.e. the active ingredient-containing polymer samples show a substantial antimicrobial effect on the test strains used.

Comparison of the samples of material with one another shows no noteworthy or tendency towards a better antibacterial effect of one material. The individual susceptibility of each bacterial strain is evident in the comparison of the test strains with one another. It was possible to demonstrate that the samples of material were active against pathogens of urinary tract infections.

No zones of inhibition, and thus no antibacterial activity either, were found with the three materials from the comparative experiments of Examples 26, 27 and 28.

### Long-Term Activity Over 30 Days

The tests were repeated after 30 days and gave the same result.

### Active Ingredient Release

In a dynamic test system, a nutrient solution with about 100 microbes/ml was continuously pumped over an active ingredient-containing silicone elastomer from Example 11 at a flow rate of 0.4 ml/min. The total volume of the nutrient solution present in the test system was 16 ml. Every 24 hours, 4 ml of solution were discharged and replaced by 4 ml of new nutrient solution with about 100 bacteria/ml. The ciprofloxacin concentration in each of the 4 ml of solutions removed was determined by HPLC. After 30 days, the test specimen was removed, cautiously rinsed and cut into three pieces: one piece was immediately rubbed on a sterile agar plate. The second piece was briefly shaken in sterile saline solution, and then the rinsing liquid was likewise plated out on an agar plate. The third piece was treated with ultrasound in sterile saline solution.

The agar plates were incubated at 37° C. for 20 hours. After the incubation, the agar plates were checked for bacterial colonies and the number was counted. Only 10 colonies were counted on the agar plate on which the piece of elastomer had only been rubbed. The cause may in this case be bacteria which have not been washed off on rinsing. No bacterial colonies grew on the agar plates of the other pieces.

The cipro-containing silicone elastomer thus had adequate surface protection, so that no bacteria were able to adhere to the surface.

In the measurement of the total amount of ciprofloxacin released from the piece of silicone elastomer during the 30-day duration of the experiment it was particularly surprising that only about 5% of the total amount of cipro contained in the piece were released. It is possible to conclude from this that the piece of silicone elastomer will have very good activity over a distinctly longer period than 30 days too.

Contrary to the teaching of EP 688 564 (page 6, line 32 to 36), according to which only readily water-soluble active ingredients are efficiently released from the silicone elastomer matrix, it was possible here to demonstrate clearly that the ciprofloxacin betaine with a low water solubility of only about 0.4 g/litre also provides very good protection of the catheter surface from bacterial adhesion.

1. An antimicrobial composition comprising a) at least one silicone elastomer and b) at least one antimicrobial active compound homogeneously distributed therein.

2. A composition according to claim 1, wherein the at least one antimicrobial active compound is a quinolone or a physiologically tolerated salt or a quinolone.

3. A composition according to claim 2, wherein the quinolone or physiologically tolerated salt of a quinolone is ciprofloxacin or a physiologically tolerated salt of ciprofloxacin.

4. A shaped article comprising a composition according to claim 1.

5. A shaped article according to claim 4, which is a catheter.

6. A shaped article according to claim 5, which is a urinary tract catheter.

7. A shaped article according to claim 5, which is a central venous catheter.

8. A process for preparing a composition according to claim 1, comprising combining at least one silicone elastomer and at least one antimicrobial active compound and mixing in such a way that the at least one antimicrobial active compound becomes homogenously distributed therein.

9. A process for preparing a shaped article, comprising providing a composition according to claim 1, and shaping said composition into said shaped article.

10. A method comprising introducing a shaped article according to claim 6 into the urinary tract of a patient in need thereof.

11. A method comprising introducing a shaped article according to claim 7 into a vein of a patient in need thereof.

12. A composition according to claim 1, wherein said active compound is incorporated in said elastomer in the form of a suspension.

13. A composition of claim 12, wherein the average particle size d₅₀ of from 0.5 to 15 μm.

14. A composition of claim 13, wherein the particle size distribution is from 0.1-30 μm.

15. A composition according to claim 1, wherein the particle size of the elastomer is from 0.1-30 μm.

16. A composition according to claim 1, wherein said active compound is water soluble.

17. A composition according to claim 12, wherein said active compound is an antimicrobially active substance capable of suppressing the colonization of organisms.

18. A shaped article according to claim 4, produced by cross linking a silicone rubber formulation at a temperature from 150-300° C.

19. A shaped article according to claim 18, wherein said cross linking comprises platinum catalyzed formulations that retain activity when said active compound comprises at least one amine group.

20. A composition according to claim 12, wherein a suspending medium is chemically incorporated into the silicone elastomer.
